(19) United States
(12) Patent Application Publication
(10) Pub. No.: US 2009/0030281 A1
(43) Pub. Date: Jan. 29, 2009

(54) APPARATUS SUITED FOR USE IN A SPACE WHICH IS DIFFICULTLY ACCESSIBLE

(75) Inventor: Paulus Breedveld, Gouda (NL)

Correspondence Address: PEACOCK MYERS, P.C., 201 THIRD STREET, N.W., SUITE 1340, ALBUQUERQUE, NM, 87102, US

(73) Assignee: Technische Universiteit Delft, Delft (NL)

(21) Appl. No.: 12/178,085

(22) Filed: Jul. 23, 2008

Related U.S. Application Data
(63) Continuation-in-part of application No. PCT/NL2007/050030, filed on Jan. 25, 2007.

(30) Foreign Application Priority Data
Jan. 30, 2006 (NL) 1031031

(51) Int. Cl. A61B 1/005

(52) U.S. Cl. 600/139; 606/1

(57) ABSTRACT

An apparatus suitable for use in a space that is not easily accessible, comprising a distally provided head, which can be equipped with an inspection organ and/or intervention organ, and which is designed to be introduced into a cavitous body, a proximal control member remaining permanently outside the body, and a connecting body extending between the control member and the distal head, wherein the connecting body is designed for being at least partly introduced into the body, and wherein the head is provided with at least one rotatable bypass organ for at least one coupling element, a first end of which at least one coupling element is coupled to a permanently fixed point and a second end is position-variable, and wherein the first and the second ends of the coupling element are located at the side of the control member, wherein the connecting body is not compressible and surrounds the at least one coupling element, acting as guide.

### CROSS-REFERENCE TO RELATED APPLICATIONS

This application is a continuation-in-part application of Patent Cooperation Treaty Application Serial No. PCT/NL2007/050030, entitled “Apparatus Suited for Use in a Space Which is Difficultly Accessible”, to Technische Universiteit Delft, inventor is Paulus Breedveld, filed on Jan. 25, 2007 and the specification and claims thereof are incorporated herein by reference.

This application claims priority to and the benefit of the filing of Netherlands Patent Application Serial No. 1031031, entitled “Apparatus Suited for Use in a Space Which is Difficultly Accessible”, filed on Jan. 31, 2006, the specification and claims thereof are incorporated herein by reference.

### STATEMENT REGARDING FEDERALLY SPONSORED RESEARCH OR DEVELOPMENT

The research culminating in the present invention was made possible by a grant from the Royal Netherlands Academy of Arts and Sciences.

### INCORPORATION BY REFERENCE OF MATERIAL SUBMITTED ON A COMPACT DISC

Not Applicable.

### COPYRIGHTED MATERIAL

Not Applicable.

### BACKGROUND OF THE INVENTION

1. Field of the Invention (Technical Field)

The invention relates to an apparatus suitable for use in a space that is not easily accessible, comprising a distally provided head, which can be equipped with an inspection organ and/or intervention organs and which is designed to be introduced into a cavitous body, a proximal control member remaining permanently outside the body, and a connecting body extending between the control member and the distal head, wherein the connecting body is designed for being at least partly introduced into the cavitous body.

2. Description of Related Art

Such an apparatus is useful for examining an industrial installation, for example, the steam pipes of a reactor. An example of such an apparatus equipped with an inspection organ is known from the international patent application PCT/NLOO/00163.

Such an apparatus is also useful, albeit in a greatly miniaturized form, as instrument for medical examination. Such an instrument is described in the international patent application PCT/NL05/000564, the contents of which are herewith considered to be included by reference.

### BRIEF SUMMARY OF THE INVENTION

Both the apparatus to be used in industry and the apparatus for medical application may, complementary to, or instead of the inspection organ, be equipped with an intervention organ. Where a medically applicable apparatus is concerned, such an intervention organ serves, for example, for performing a surgical operation or the very exact introduction of medication. Another example of a medical intervention organ is the generally known gripping instrument for performing a biopsy. With an industrially applied apparatus, the intervention organ may be used for repair work.

In the case of industrial inspection systems according to the prior art, the propulsion of the head of the apparatus in the cavitous body to be examined or treated is provided by electromagnetic driving means. With medical (inspection) systems, such as the usual flexible endoscopes, gastro-scopes or colonoscopes the head has to be displaced by proximally pushing the connecting body forward instead the head being provided with a drive element. A drawback of this is that the head may catch behind protuberances inside the patient, which may cause the connecting body of the scope to buckle as a result of the pushing force. This may be painful for the patient and may cause internal injury.

From the American patent specification U.S. Pat. No. 5,571,114 an apparatus according to the preamble is known, wherein the head is provided with at least one rotatable bypass organ for at least one coupling element, a first end of which, is coupled to a permanently fixed point and a second end is position-variable, and wherein the first and the second ends of the coupling element are located at the side of the control member.

In the apparatus known from the American patent specification U.S. Pat. No. 5,571,114, said coupling element is provided in the form of a chain or strip reciprocally moving at the outside of and along the connecting body that extends between head and control member, which connecting body may contain, for example, a fibre glass bank.

By driving the rotatable bypass organ located near the head of the known apparatus, the connecting body of the known apparatus that is coupled to an end of the chain or strip next to it is moved inside the body to be examined. In order to drive the bypass organ, a drive shaft extending in the longitudinal direction of the connecting body is required.

A drawback of the known apparatus is the relative complexity of the construction which is maintenance-prone and, owing to the use of the broad chain or strip, it is flexurally relatively stiff in the transverse direction. As a result, the known apparatus is not particularly suitable for practical applications, in particular where medical application is concerned.

### BRIEF DESCRIPTION OF THE SEVERAL VIEWS OF THE DRAWINGS

In the drawing,

FIG. 1 schematically shows an apparatus, with the omission of some of the components that are of little importance with respect to explaining the invention.

### DETAILED DESCRIPTION OF THE INVENTION

The object of the invention is to provide an apparatus that can be moved in a reliable manner through the inside of the cavitous body to be inspected or treated.

A further object of the invention is to avoid using the standard electromagnetic drive in the head.

Still another object of the invention is to provide an apparatus that is simple to clean and, if necessary, disinfect.

Still another object of the invention is to provide an apparatus that can be readily miniaturized.

Still another object of the invention is to provide an apparatus that is well suited to be used in aggressive environments caused, for example, by high temperatures, strong chemicals or radioactivity. Underwater applications involving difficult conditions (high water pressure), form an area of application for which the apparatus according to the invention intends to find a solution.

To this end the apparatus according to the invention is characterized by one or several of the appended claims.

In a first aspect of the invention, the apparatus is characterized in that the connecting body is not compressible and surrounds the at least one coupling element, acting as guide, in that the at least one bypass organ is designed to provide traction along a wall of the body, and that the control member is adjustable in relation to the permanently fixed point, in order to displace the at least one coupling element in the longitudinal direction inside the connecting body in such a manner, that the bypass organ providing the traction begins to rotate.

This enables the bypass organ to move the head along the wall of the body without the risk that the movement of the control member will cause the connecting body to buckle.

It is also an advantage that the traction-providing bypass organ does not comprise electromechanical means for realizing the displacement, so that the apparatus can be manufactured at low costs and is suitable for simple cleaning and disinfection. It is also extremely simple to miniaturize the apparatus according to the invention. Due to the low manufacturing costs, the apparatus is moreover particularly suitable to be embodied as disposable, which is especially desirable in the case of medical applications.

In order to be able to follow bends in the cavitous body to be inspected or treated, it is further desirable for the connecting body to be bendable around the longitudinal axis.

It is also useful that the second end of the at least one coupling element has tensile strain-bearing capacity. This ensures that an accurate relation is maintained between the displacement of the control member and the corresponding rotation of the bypass organ.

The tensile strain-bearing capacity referred to realized in different ways, for example, by suspending a weight from the second end, as shown in the FIGURE legend included with this application. However, other variants are sometimes also advantageous, for example, a mechanical coupling between the first end and the second end of the coupling element, which maintains a fixed relation between both positions. A suitable example for this is a gearbox. A simple spring load at the second end may also suffice.

The bypass organ may be selected from various embodiments. However, it is preferred for the at least one bypass organ to be a predetermined choice from the group comprising a pulley, a track chain, a propulsion organ as described in the international patent application PCT/NL05/000564.

It is also possible to use various bypass elements that are different from each other.

In a first preferred embodiment, the apparatus is characterized in that a series of bypass organs is provided arranged in the form of a star around a longitudinal axis of the apparatus.

A second preferred embodiment of the apparatus according to the invention is characterized, in that a succession of bypass elements is provided along the longitudinal axis of the apparatus.

Another aspect of the invention relates to the embodiment of the apparatus that is characterized in that there are at least two bypass organs, of which at least one bypass organ is placed on its own hinged arm.

This makes it possible in a simple manner to use the apparatus with cavity-like bodies of varying diameters.

It is furthermore desirable for at least one of the arms of the bypass organs to have a preferential position such that the bypass organs incline away from each other. This ensures a continuous contact of the bypass organs with the wall of the cavitous body to be inspected or treated.

Hereinafter the invention will be further elucidated by way of a schematic exemplary embodiment as shown in the drawing illustrating the working principle of the apparatus according to the invention.

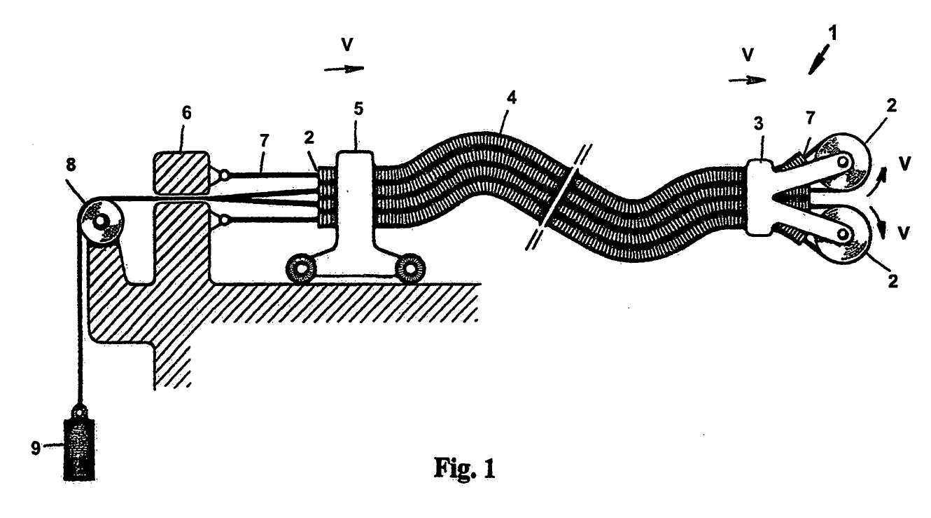

FIG. 1 shows that the apparatus possesses a distally placed head 1. This head 1 may be equipped with an inspection organ and/or intervention organ (not shown) and is designed to be introduced into a cavitous body to be inspected or treated. In industrial applications this cavitous body may, for example, be the inside of steam pipes of a boiler or reactor. In the case of medical applications this may, for example, be an intestine.

At the proximal side and intended to remain permanently outside of the body to be inspected or treated, a control member 5 is provided. A connecting body 4 extends between the control member 5 and the head 1, connecting the control member 5 and the head 1. Said connecting body 4 is also designed for being at least partly introduced into the cavitous body to be inspected or treated.

In accordance with the invention, the connecting body 4 is not compressible and serves also as housing for the at least one coupling element 7 extending through the connecting body 4. As such the connecting body 4 acts as guide for the at least one coupling element.

In the case illustrated, two coupling elements 7 are shown in the form of cables running through the connecting body 4 from outside the control member 5 to the head 1, and from the head 1 back to outside the operating member 5.

In order to facilitate the reciprocal movement of the cables 7, the head is provided with at least one, in the illustrated case with two, bypass organs 2. These bypass organs 2 are designed to provide traction along a wall of the body and in order to achieve this traction, the cables 7 are at a first end coupled with a permanently fixed point 6, while a second end is position-variable. In the case shown, this second end is provided with a weight 9 and the cables 7 run over a pulley 8, the gravitational force keeping the cables 7 under tension strain. As has been explained above, it is also possible to apply other methods in order to produce this tension strain.

FIG. 1 shows that both the first and the second end of the cables 7 are located at the side of the control member 5, outside the connecting body 4.

It is further of importance that in order to displace the cables 7 running through the connecting body 4 in the longitudinal direction, the control member 5 can be adjusted in relation to the permanently fixed point 6 by hand or by means of a motor drive. As a consequence of this displacement, the pulleys 2 begin to rotate and, as these pulleys are coupled with the wall of the cavitous body to be inspected or treated, they ensure that at the head side FIG. 1 a propelling force is applied to the apparatus.

The workings of the construction may be illustrated as follows.

By displacing the control member 5 in the direction of arrow V the upper and lower cable 7 is pulled out of the connecting body 4. Owing to the connecting body 4 not being compressible, the upper and lower cable 7 is pulled inward at the side of the head 1. Because the cables 7 are guided around the pulleys 2, said pulleys 2 are caused to rotate at a rotational speed that corresponds with the displacement speed of the control member 5. In the absence of slip between the pulleys 2 and the wall of the cavitous body to be inspected or treated, the head 1 will correspondingly move at the same speed as the control member 5, so that pushing forces on the connecting body 4 are avoided and there is no risk of the connecting body 4 buckling.

Although FIG. 1 shows that two pulleys 2 are used, this number may be increased as desired up to a series of pulleys arranged, for example, in the shape of a star around the longitudinal axis of the connecting body 4.

It is also possible to arrange along the longitudinal axis of the connecting body 4 several pulleys in succession.

It will be further obvious to the person skilled in the art that the connecting body 4 is preferably bendable around the longitudinal axis, which, owing to the construction of the apparatus according to the invention involves no risk of the connecting body 4 buckling. However, this flexibility does make it possible to easily follow curves in the cavitous body to be inspected or treated. Although FIG. 1 shows a pulley being used as bypass organ 2, it is also possible to use for this purpose a track chain or a propulsion organ as described in the international patent application PCT/NL05/000564, the contents of which are herewith considered to be included by reference.

It is further observed that FIG. 1 shows that the bypass organs 2 used are each mounted on a rigid arm. Within the scope of the invention it is also useful and even advantageous to embody at least one of these arms, and preferably both arms, to be hinged in such a manner that said arms have a preferential position in which the bypass organs 2 incline away from each other. This renders it easy to follow the diameter variations of the cavitous body to be inspected.

What is claimed is:

1. An apparatus suitable for use in a space that is not easily accessible, comprising:
a distally provided head, that can be equipped with an inspection organ and/or intervention organ, and which is designed to be introduced into a cavitous body;
a proximal control member remaining permanently outside the body; and
a connecting body extending between the control member and the distal head; and
wherein the connecting body is designed for being at least partly introduced into the body; and
wherein the head is provided with at least one rotatable bypass organ for at least one coupling element, a first end of which at least one coupling element is coupled to a permanently fixed point and a second end is position-variable; and
wherein the first and the second ends of the coupling element are located at the side of the control member, and the connecting body is not compressible and surrounds the at least one coupling element, thereby acting as guide; and
wherein the at least one bypass organ is designed to provide traction along a wall of the body; and
wherein the control member is adjustable in relation to the permanently fixed point, in order to displace the at least one coupling element in the longitudinal direction inside the connecting body in such a manner that the bypass organ providing the traction begins to rotate.

2. An apparatus according to claim 1, wherein the first and the second end of the coupling element are located outside the connecting body.

3. An apparatus according to claim 1, wherein the connecting body is bendable around the longitudinal axis.

4. An apparatus according to claim 1, wherein the second end of the at least one coupling element is under tensile strain.

5. An apparatus according to claim 1, wherein the at least one bypass organ is a predetermined choice from the group consisting of a pulley, a track chain, and a propulsion organ.

6. An apparatus according to claim 1, wherein a series of bypass organs are provided arranged in the form of a star around a longitudinal axis of the apparatus.

7. An apparatus according to claim 1, wherein a succession of bypass elements are provided along the longitudinal axis of the apparatus.

8. An apparatus according to claim 1, wherein there are at least two bypass organs, of which at least one bypass organ is placed on its own hinged arm.

9. An apparatus according to claim 8, wherein at least one of the arms of the bypass organs has a preferential position such that the bypass organs incline away from each other.
